(19) United States
(12) Patent Application Publication
(10) Pub. No.: US 2008/0255598 A1
(43) Pub. Date: Oct. 16, 2008

(54) Lancing Device With Replaceable Multi-Lancet Carousel

(75) Inventors: Richard W. LeVaughn, Newnan, GA (US); Stephen J. Flynn, Peachtree City, GA (US); Jason R. Heath, Marietta, GA (US); Christopher J. Ruf, Marietta, GA (US); Avi M. Robbins, Longwood, FL (US); Stephanie J. Campbell, Kennesaw, GA (US); Gwenn E. Kennedy, Ellenwood, GA (US); Carl E. Griffin, Marietta, GA (US); John C. Irwin, Woodstock, GA (US); Mary Kate Pynes, Dallas, GA (US); Mitchell A. Solis, Cumming, GA (US)

Correspondence Address: GARDNER GROFF GREENWALD & VILLANUEVA. PC, 2018 POWERS FERRY ROAD, SUITE 800, ATLANTA, GA, 30339, US

(73) Assignee: Facet Technologies, LLC, Marietta, GA (US)

(21) Appl. No.: 10/569,032

(22) PCT Filed: Aug. 19, 2004
(86) PCT No.: PCT/US2004/026971

§ 371 Date: Jun. 26, 2008

Related U.S. Application Data
(60) Provisional application No. 60/496,844, filed on Aug. 20, 2003.

(51) Int. Cl. A61B 17/32

(52) U.S. Cl. 606/183; 606/181

(57) ABSTRACT

A housing having an actuator mechanism and a replaceable carousel having a series of lancets. The carousel is removably received in the housing and the actuator mechanism is operable to actuate the individual lancets. In exemplary embodiments the lancing device has, amongst other features, key and detent mechanisms for orienting and securing the carousel in the housing, tangentially configured lancets for compactness, actuator alignment features for aligning the actuator with the lancets, charging mechanisms for ease in charging the lancets for launching, and shutter mechanisms for retaining the lancets in the housing after launching. Also included is a method for lancing using a lancing device according to the invention.

### CROSS-REFERENCE TO RELATED APPLICATIONS

This application claims the priority benefit of U.S. Provisional Patent Application Ser. No. 60/496,844, filed Aug. 20, 2003, the content of which is hereby incorporated herein by reference in its entirety.

### TECHNICAL FIELD

The present invention relates generally to medical devices and procedures and, more particularly, to lancing devices for collecting samples of blood or other bodily fluid.

### BACKGROUND OF THE INVENTION

Many medical procedures require puncturing of the skin, and sometimes underlying tissues, of an animal or human subject. For example, a sharp lancet tip is commonly used to puncture the subject's skin at a lancing site to obtain a sample of blood, interstitial fluid or other body fluid, as for example in blood glucose monitoring by diabetics, and in blood typing and screening applications. In some instances, a person must periodically sample their blood for multiple testing throughout the day or week. Because re-use of a lancet can result in infection or spread of bloodborne contaminants, persons requiring repeated testing often must carry multiple lancets with them. This can be inconvenient and lead to reduced compliance with a prescribed test regimen. Accordingly, it can be seen that needs exist for a convenient, compact multi-use lancing device.

Various devices are known for sampling blood and other body fluids for analysis of the condition of a human or other animal subject. For example, U.S. Pat. No. 5,971,941 is understood to show a cassette with test strips for placement by a slider. A lancet pierces the skin surface so that blood can be obtained for analysis. The lancets are integrated on a test strip, and are positioned together with the test strip. Another embodiment is understood to show a disposable cylindrical insert having a lancet and a test membrane with an aperture for the lancet. The insert is inserted in a mounting cavity of a plunger or piston, which forces the lancet outward for blood withdrawal. DE 198 19407 A1 is understood to show a multiplicity of test strips with integrated lancets for insertion into an analysis device.

U.S. Pat. No. 4,787,398 is understood to show a device with a plunger for directing a lancet outward, and has an evaluation system and a display system. A replaceable unit is applied to the device for each measurement. The replaceable unit comprises the lancet and a test strip, which is wetted with blood. This replaceable unit is thrown away after each use. EP 0 449 525 A1 is understood to show a blood withdrawal system wherein a new lancet is inserted manually into a release device before each use. A test strip is then inserted into the device. U.S. Pat. No. 4,627,445 is understood to show a device for measuring blood sugar, with an integrated blood withdrawal unit. A new replaceable lancet and test elements must be installed to the device for testing, and afterward disassembled. U.S. Pat. No. 5,951,492 is understood to show a disposable unit with a capillary tube and a test strip, to which sampled blood taken is applied. The capillary tube includes a lancet. A new disposable unit is attached and removed before and after each measurement.

EP 0877250 A2, EP 0949506 A2 and EP 811843 A2 are understood to show devices having a multiplicity of test elements arranged on a rotatable disk carrier. The test elements are brought successively into a working position and pushed out of the housing to be wetted with blood. U.S. Pat. No. 6,228,100 and U.S. Pat. No. 4,794,926 are understood to show lancets arranged on a carrier, which is rotated with respect to a housing.

German Application DE 100 57 832 C1 is understood to show a lancing device of a known form. Other lancing devices understood to include multiple lancets are shown, for example, in U.S. Patent App. Serial No. 2002/0087056 A1 and WO 02/36010 A1. EP 0589186 B1 is understood to show a lancet with a removable protective cap. WO 01/66010 A1 is understood to include a multiplicity of lancets in a magazine, with an opening of the chamber closed by an elastic material, which is penetrated in the puncture process.

Known sampling devices have, however, not proven fully satisfactory to all users for a variety of reasons. Accordingly, it is to the provision of an improved sampling device that the present invention is primarily directed.

### SUMMARY OF THE INVENTION

Generally described, the present invention includes several innovative features of a lancing device and a replaceable multi-lancet carousel for use therewith. In the embodiments described herein, the carousel has a carrier and a series of lancets that are carried by the carrier. The lancets each have a body and a puncturing tip projecting from the body. And the lancing device has a housing that holds the carousel so that the user can easily remove and replace carousels as needed. Several of the innovative features can, however, be implemented on a single-use lancing device, if desired.

In one aspect, the invention is a lancing device that includes at least one shutter mechanism. Preferably, there is one shutter mechanism coupled to the carrier for each lancet. The shutter mechanisms each include a shutter that moves between an open position not retaining the lancet from moving to a puncturing position and a closed position retaining the lancet in a retracted position. In the puncturing position, the lancet tip sticks out of the housing, and in the retracted position, the lancet tip is returned to safely within the housing. The shutter moves from the open position to the closed position in response to the lancet being actuated to puncture the skin. Preferably, the shutter mechanism includes a shutter release member that moves from an engaged position where it holds the shutter in the open position, to a release position not retaining the shutter in the open position when the lancet is rotationally actuated to lance the skin.

In another aspect, the invention is a lancing device that includes at least one charging mechanism. Preferably, there is one charging mechanism for each lancet. The charging mechanisms each have two or another number of loading recesses and two or another number of loading tabs that are rotated into and retained in the loading recesses when the lancet is moved from a charged position to a charged and set position. In the charged position, the lancet is charged for launching, and in the charged and set position, the lancet is charged and retained in place. In addition, the loading tab is rotated out of the loading recess when the lancet is moved from the charged and set position to the charged position, which frees the lancet for launching to the puncturing position.

In yet another aspect, the invention is a lancing device that includes at least one actuator mechanism. Preferably, there is one actuator mechanism rotationally coupled to the carrier for actuating all of the lancets. The actuator mechanism includes an actuator arm that can be operably aligned with and moved into engagement with each of the lancets to rotate the lancets from the charged and set position to the charged position, thereby freeing the lancets to be launched. Preferably, there is a cam surface on the lancets or on the actuator arm that is configured to cause the lancets to rotate when they are engaged by the actuator arm.

In still another aspect, the invention is a lancing device that includes a carousel with a carrier that is adapted for carrying the lancets in a non-radial, preferably a tangential, configuration. In addition, the housing preferably has a generally wedge-shaped protrusion corresponding to each lancet, with the protrusions each having a surface generally perpendicular to a puncturing stroke direction of a corresponding one of the lancets, and the housing having lancet tip openings in those perpendicular surfaces. Furthermore, the lancing device preferably has at least one lock mechanism including at least one key-tabbed member and at least one keyed opening that matingly receives the key-tabbed member so that the carousel can be rotated into a locked position in which it is secured to the housing.

In still another aspect, the invention is a lancet carousel that is replaceable in the housing of a lancing device. The carousel includes a plurality of lancets and a carrier that carries the lancets in a non-radial, preferably a tangential, configuration. Preferably, the carousel has at least one shutter mechanism including at least one shutter that moves between an open position not retaining the lancet from moving to the puncturing position and a closed position retaining the lancet in the retracted position. In addition, the carousel preferably has at least one charging mechanism including at least one loading recess and at least one loading tab that rotates into and is retained in the loading recess when the lancet moves from the charged position to the charged and set position. Furthermore, the carousel preferably has at least one actuator mechanism including at least one actuator arm that moves into engagement with the lancets to rotate the lancets from the charged and set position to the charged position to free the lancets for launching.

And in yet another aspect, the invention is a method of lancing skin, preferably using a lancing device and/or lancet carousel including one or more of the innovative features described herein. An example method includes the steps of providing a lancing device having a housing and a lancet carousel, the lancet carousel including a plurality of lancets carried by a carrier in a non-radial configuration. In addition, the method includes the steps of inserting the carousel in the housing, charging one of the lancets, launching the charged lancet, advancing the actuator mechanism from one of the lancets to a next one of the lancets, repeating these steps until all the lancets are actuated, and removing the carousel. The step of charging one of the lancets may include, for example, grasping a sterility cap of one of the lancets, pushing the lancet inward so that at least one loading tab is guided by a track to guide the lancet to a charged position, and rotating the lancet so that the loading tab is received in at least one loading recess to retain the lancet in a charged and set position. The step of launching the charged lancet may include, for example, actuating an actuator mechanism to rotate the lancet back to the charged position so that the loading tab is removed from the loading recess and the charged lancet is free to be launched. And the step of advancing the actuator mechanism may include, for example, rotating an actuator arm of the actuator mechanism from operable alignment with the one lancet to operable alignment with the next lancet.

Accordingly, it can be seen that the present invention, in its various aspects as described herein, provides ease of use in a compact carrying unit to lessen the difficulty and inconvenience associated with typical lancing equipment. With these improvements, users are more likely to stick with a blood testing regimen to monitor their health conditions.

### BRIEF DESCRIPTION OF THE DRAWINGS

FIG. 1 is a perspective view of a lancing device according to an exemplary embodiment of the present invention, with the device in an open position to show a housing having a top section, a bottom section, and an actuator mechanism, and a replaceable carousel having a series of lancets.

FIG. 2 is a perspective view of the lancing device of FIG. 1 in a closed position.

FIG. 3 is a plan view of the inside of the housing top section of the lancing device of FIG. 1.

FIG. 4 is a perspective view of the housing top section of FIG. 3.

FIG. 5 is a plan view of the outside of the housing top section of the lancing device of FIG. 1.

FIG. 6 is a plan view of the inside of the housing bottom section of the lancing device of FIG. 1.

FIG. 7 is a perspective view of the housing bottom section of FIG. 6.

FIG. 8 is a perspective view of a housing bottom section according to an alternative embodiment of the present invention.

FIG. 9 is a perspective view of the lancing device of FIG. 1 in the open position, showing the lancet carousel loaded onto the housing bottom section.

FIG. 10 is a perspective view of the lancing device of FIG. 1 in the open position, showing the lancet carousel loaded onto the housing top section.

FIG. 11 is a perspective view of the lancet carousel of the lancing device of FIG. 1, showing a shutter mechanism for retaining the lancet in the housing after launching.

FIG. 12 is a plan view of the lancet carousel of FIG. 11.

FIG. 13 is a plan view of the lancet carousel of FIG. 11, showing the charging of the lancets.

FIG. 14 is a perspective view of the lancet carousel of FIG. 13.

FIG. 15 is a plan view of the lancet carousel of FIG. 11, showing the charging of the lancets.

FIG. 16 is a perspective view of the lancet carousel of FIG. 15.

FIG. 17 is a perspective view of the actuator mechanism of the lancing device of FIG. 1.

FIG. 18 is an elevation view of the actuator mechanism of FIG. 17.

FIG. 19 is a plan view of the actuator mechanism of FIG. 17.

FIG. 20 is a cross sectional view of the actuator mechanism taken at line 20-20 of FIG. 19.

FIG. 21 is a perspective view of one of the lancets of the lancet of FIG. 1.

FIG. 22 is an end view of the lancet of FIG. 21.

FIG. 23 is an end view of the lancet of FIG. 21 and a cross sectional view of the actuator mechanism taken at 20-20 of FIG. 19, showing the actuating of the lancet:

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be unnecessarily limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

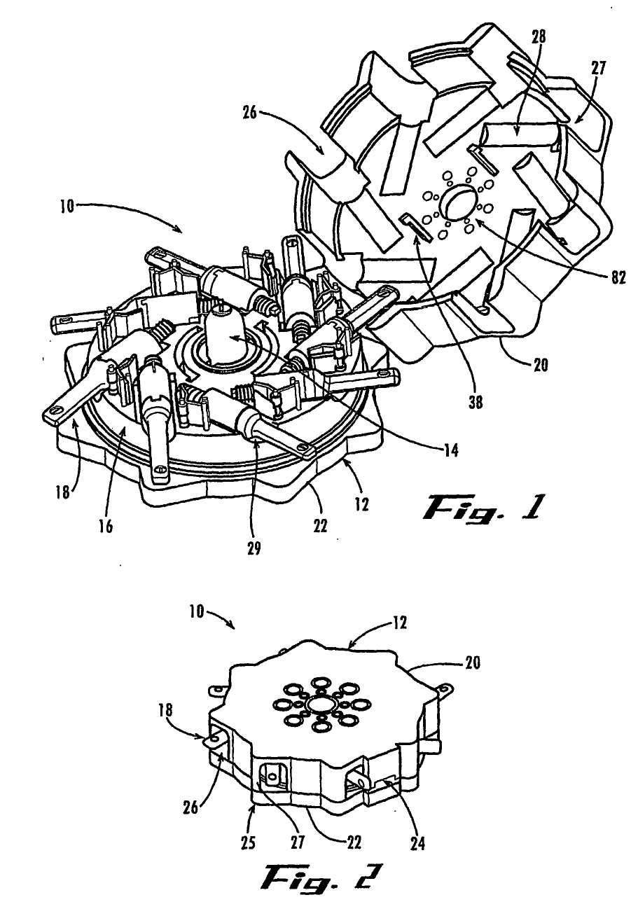

With reference now to the drawing figures, FIGS. 1 and 2 show a lancing device 10 according to an exemplary embodiment of the present invention. The lancing device 10 includes a housing 12 having an actuator mechanism 14 and a lancet carousel 16 having a series of lancets 18 stored on a carrier 17. The housing 12 is reusable and the lancet carousel 16 is replaceable so that a used one can be easily removed from and a fresh one inserted into the housing. The lancets 18 each include a body and a puncturing member (e.g., a needle or blade) extending from the body, with the puncturing member having a sharp tip for puncturing the skin. The number of lancets 18 included is selected based on the desired size of the device 10 and also on the number of times sampling is typically needed in a given period of time.

In a typical commercial embodiment, the housing 12 has a first or top section 20 and a second or bottom section 22 that are hingedly coupled together in a clamshell arrangement for movement between the open position of FIG. 1 and the closed position of FIG. 2. Preferably, the housing 12 includes a spring-biased hinge 24 for urging the sections open and a snap-tight closure for releasably securing the sections closed together. In alternative embodiments, the housing has a twist-lock or other closure, a hinge without a spring, a spring-biased hinge for urging the sections closed, and/or a swivel, pivotal, or other coupling. And in another embodiment, the housing is a solid piece with a side slot or other opening for removing and inserting the carousels.

In addition, the housing 12 has at least one opening 26 through which the lancets 18 extend to a fully extended puncturing position for puncturing the skin. Preferably, there are a plurality of the openings with a dedicated opening 26 for each lancet 18, and the actuator mechanism 14 rotates into alignment with each lancet for launching it through its opening. Alternatively, the housing may have only one opening (or another number of openings) for all the lancets and a rotary carousel for sequentially aligning the lancets with the opening.

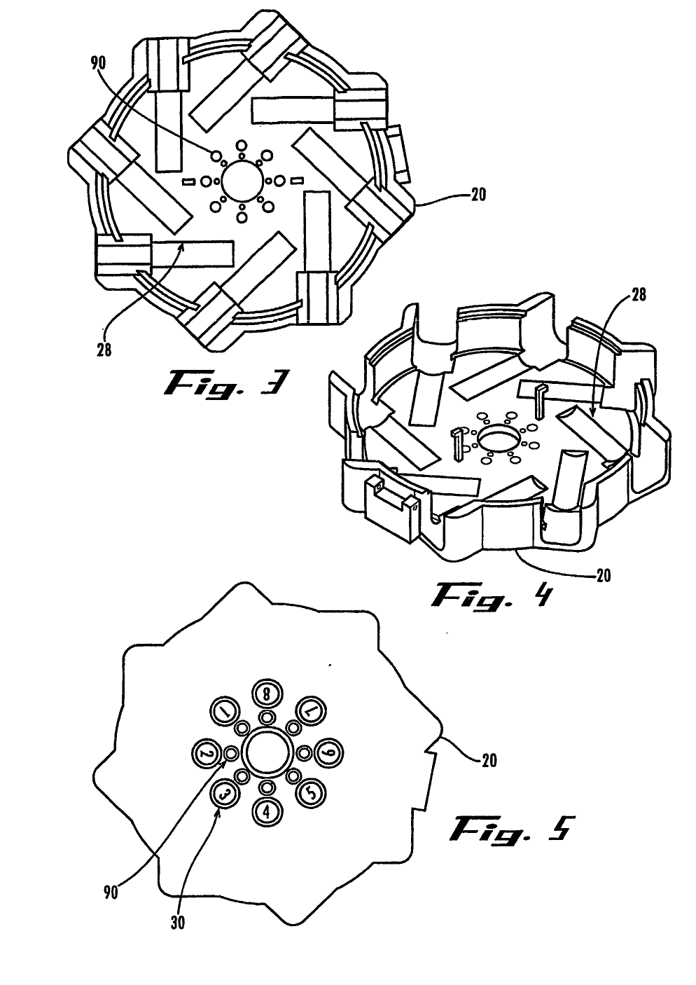

Referring additionally to FIGS. 3-5, there are illustrated several features that may be included in the top section 20 of the housing 12. As shown in FIGS. 1, 3, and 4, the housing top section 20 may have guide recesses 28 for receiving the lancets 18, and loading recesses 27. Preferably, the guide recesses 28 cooperate with recesses 29 in the carousel 17 to provide guidance for the lancets 18, and the loading recesses 27 cooperate with loading recesses in the carrier for receiving loading tabs to retain the lancets in a charged and set position. Because the housing 12 is reusable, it may be desirable to manufacture it of a more durable material and to tighter tolerances, so including lancet guide recesses 28 in the housing 12, instead of in a disposable carousel 17 cover, may be desirable. In addition, as shown in FIGS. 2 and 5, the top section 20 may have indicia 30 such as numbers marked on it for indicating which lancet 18 the actuator mechanism 14 is aligned with for actuating. When including either of these features, is it preferable to provide for proper orientation of the lancet carousel 16 in the housing 12. To accomplish this, one or more of the lancets 18 may be coded to match one or more positions on the housing 12. For example, the lancets 18 and housing 12 can be color-coded or provided with matching numbers or other indicia for facilitating properly orienting the carousel 16 in the housing 12.

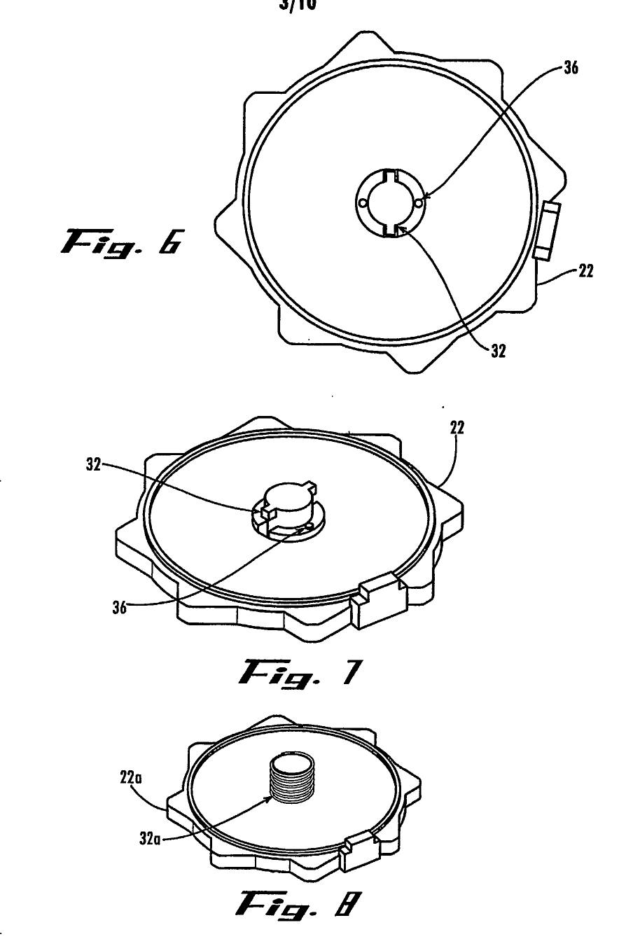

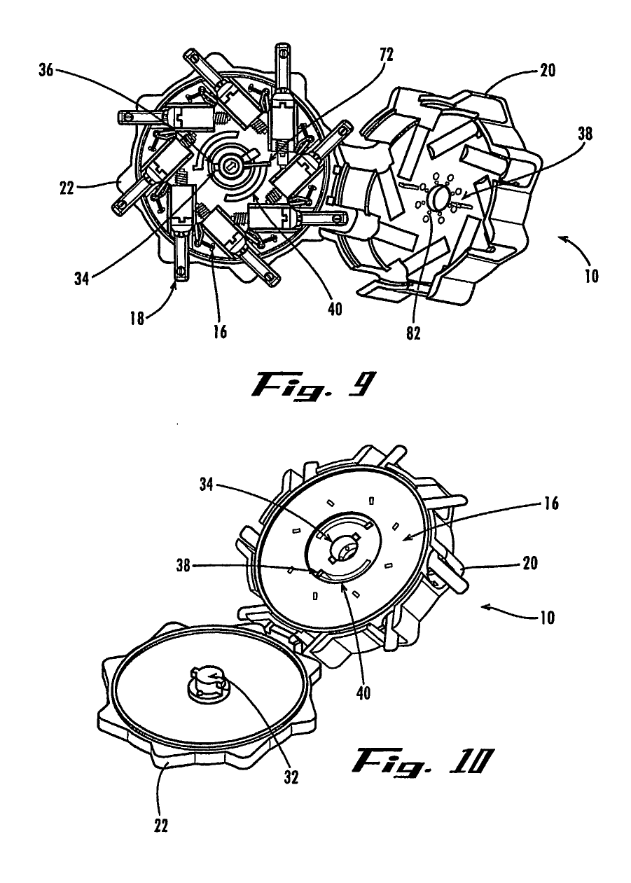

Referring now to FIGS. 6, 7, 9, and 10, the lancing device 10 may additionally or alternatively have a lock mechanism for properly orienting and securing the carousel 16 in the housing 12. In a typical commercial embodiment, the lock mechanism includes a key-tabbed member 32 on the housing bottom section 22 that is received in a mating keyhole 34 or other keyed opening in the carrier 17. And the lock mechanism further includes a detent such as a bump 26 on the housing bottom section 22. After the carousel 16 is loaded onto the housing bottom section 22 with the key-tabbed member 32 received in the keyhole 34, the user rotates the carrier 17 until the detent bump 26 aligns with the keyhole. This secures the carousel 16 in place for use. To change out the carousel 16, the user rotates it in the opposite direction, overcoming small retaining force of the detent, until the keyhole and key-tabbed member align so that the 16 can be removed. It will be understood that the key-tabbed member and detent bump may be alternatively provided on the carrier and the keyhole formed in the housing, if desired.

In an alternative embodiment shown in FIG. 8, the lancing device has a threaded member 32a on the housing bottom section 22a that is matingly received in a threaded hole (not shown) in the carousel 17, or vice versa, for properly orienting and securing the carousel 16 in the housing. This configuration works well when including the coding feature described above for facilitating properly orienting the carousel 16 in the housing 12. In other alternative embodiments, the lancing device has other lock mechanisms for orienting and removably securing the carousel 16 to the top section or another part of the housing.

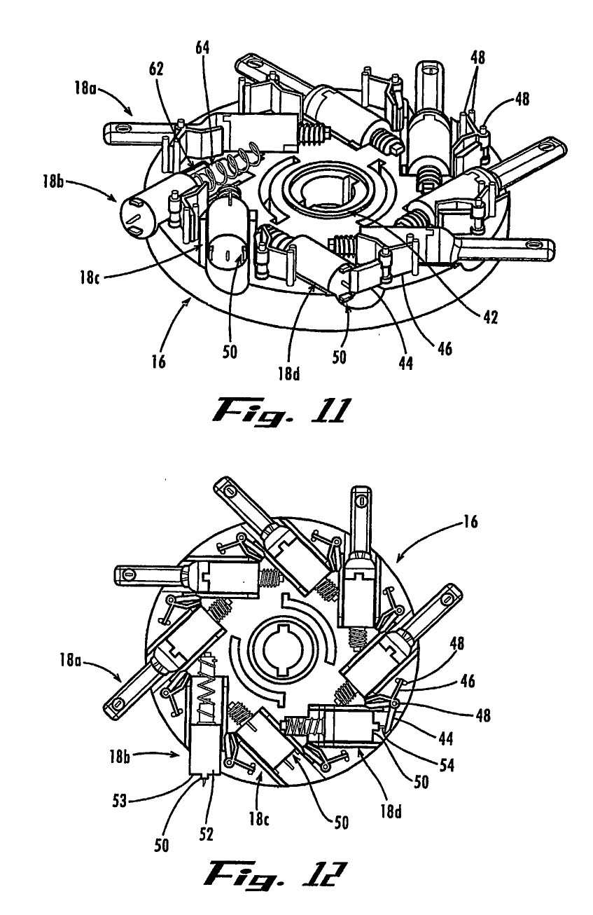

Referring still to FIGS. 9 and 10, the lancing device 10 may additionally or alternatively have a top lock mechanism for facilitating properly orienting and/or securing the carousel 16 to the housing top section 22. In a typical commercial embodiment, the lancing device 10 has a bottom lock mechanism such as that described above for orienting and securing the carousel 16 to the housing bottom section 20 for use (see FIG. 9), and additionally has a keyed or other top lock mechanism for securing the carousel 16 to the housing top section 22 after use (see FIG. 10). For example, this keyed mechanism may include two (or another number of) key-tabbed members 38 on the housing top section 20 that are received in two (or another number of) curved key-slots 40 in the carrier 17 (see also FIG. 1), or vice versa, for rotating the carousel 16 to lock it to the housing 12.

Accordingly, after the last one of the lancets 18 has been actuated, the user resets the lancing device 10 by turning the actuator mechanism 14 back to the first one of the lancets. Because all of the lancets 18 have been used, all of the lancets caps have been removed and the lancets retracted onto the housing (not shown), as will be described in more detail below. The lancing device 10 may include a structure (not shown) for rotating the carousel 16 along with the actuator mechanism 14 when resetting the actuator mechanism from the last to the first lancet 18. The carrier 17 is rotated to the position shown in FIG. 10, thereby locking the carousel 16 to the top section 20 and releasing it from the bottom section 22. The user then twists the carrier 17 to remove it from the top section 20 and to install a fresh carousel 16 onto the bottom section 22. In alternative embodiments, the lancing device has other mechanisms for orienting and removably securing the carousel to the top section or another part of the housing for use.

Referring now to FIGS. 11 and 12, there are illustrated several additional features that may be included in the lancing device 10. In these and other figures, lancets 18a, 18b, 18c, and 18d are sometimes referred to individually and, for convenience, at other times referred to collectively as “the lancets 18.” Preferably, the lancets 18 are arranged in a non-radial configuration on the carrier 17, with the carrier being generally circular. For example, the lancets 18 may be arranged in a tangential or pinwheel configuration, as shown. The lancets 18 are tangentially arranged in that the longitudinal stroke axis of the lancets is perpendicular to a radial line between the center of the carrier and the lancet axis, and the lancets are tangential to a circle whose radius is the radial line. In this way, the lancets 18 can be provided on a smaller dimensioned carrier 17, and the housing 12 can then be made correspondingly smaller.

With the lancets 18 so configured, the housing 12 preferably has a protrusion 25 for each lancet, with each protrusion 25 having a surface 27 generally perpendicular to the longitudinal puncturing stroke direction (which is thus non-radial) of a corresponding one of the lancets (see FIG. 1). The protrusions 25 are preferably wedge-shaped, and the lancet openings 26 are formed in the perpendicular surfaces 27. When using the lancing device 10, the surface 27 is placed against a skin surface such as a finger of the user.

In an alternative embodiment, the lancets are arranged in a radial configuration. In this embodiment, the carousel 16 may further include a shield member 42 that blocks the lancets 18 from interfering with the actuator mechanism 14 or the oppositely positioned lancet. It will be understood that the shield member 42 may also be included with the non-radial configured lancets 18, as is depicted in the figures.

In addition, the lancing device 10 preferably includes one or more shutter mechanisms for retaining the lancets 18 in a retracted position with the lancet tips in the housing 12 after puncturing the skin in the puncturing position. The shutter mechanisms dampen lancet vibrations, prevent lancet re-strikes after once puncturing the skin, and prevent reuse of the of the lancets after being used once. The shutter mechanisms each include at least one shutter 44 that moves from an open position in which the corresponding lancet 18 can be actuated to a closed position retaining the lancet in the housing. The shutter 44 may be a panel, arm, frame, or another flat, curved, or otherwise configured structure for retaining the lancet in the housing. Preferably, the shutter 44 moves from the open position to the closed position automatically in response to the corresponding lancet 18 being actuated to puncture the skin.

In a typical commercial embodiment, each shutter mechanism includes a spring element, for example, a torsion spring 46 mounted on posts 48 on the carrier 17, and at least one shutter release, for example, a shutter release tab 50 extending longitudinally from a front end 53 of the body 52 of the lancet 18. The spring element 46 biases the shutter 44 to rotate about the post 48 to the closed position. But the shutter release tab 50 holds the shutter 44 in the open position when the lancet 18 is in a charged and set position ready for launching to the puncturing position (e.g., see lancet 1 8c). When the user operates the actuator mechanism 14, it actuates the lancet 18 by rotating it, thereby also rotating the shutter release tab 50 from blocking the shutter 44. Preferably, the lancet 18 and the shutter 44 rotate about perpendicular axes. So now the shutter 44 presses against the lancet body 52 while the lancet 18 is launched to puncture the user's skin (e.g., see lancet 18b). But when the lancet 18 is retracted back into the housing 12, the shutter release tab 50 is no longer there to block open the shutter 44, so it swings closed to retain the lancet tip 54 in the housing (e.g., see lancet 18d).

In alternative embodiments, the shutter mechanisms include shutters that are coupled to the housing, that slide up or laterally into the closed position, and/or that are biased by coil or other types of spring elements. In these or other alternative embodiments, the shutter mechanism includes shutters that are retained in the open position by catches or other shutter release structures that are pushed, pulled, or otherwise moved out of the way by the lancet launching movement. And in these or other alternative embodiments, the shutter mechanisms include shutters that catch and retain the back or side of the lancet instead of blocking its path of travel.

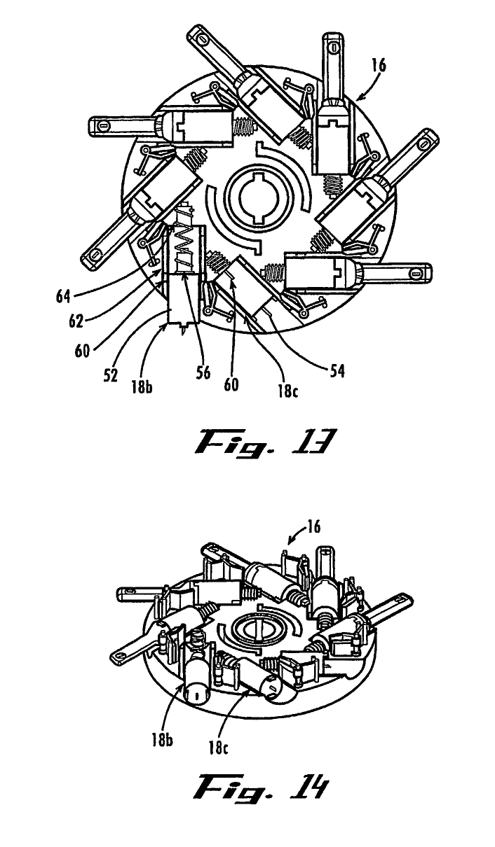

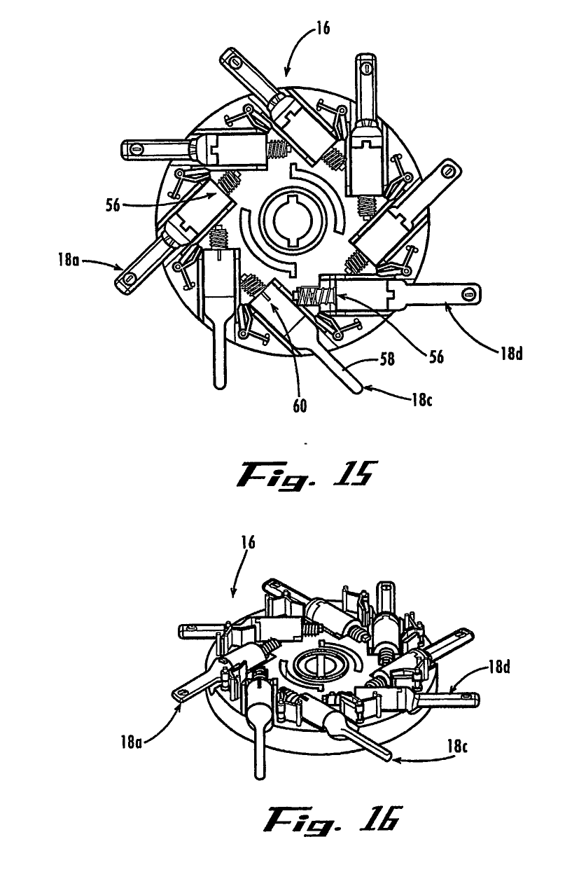

Referring now to FIGS. 13-16, there are illustrated details of the charging mechanism and the steps for charging the lancets 18 to ready them for launching. Lancet 18d of FIGS. 15 and 16 is in the rest position, with the lancet operating spring 56 in the neutral position. To charge a lancet, the user pushes it inward to the charged position of lancet 18a of FIGS. 15 and 16, thereby compressing and charging the operating spring 56. Then the user rotates the lancet, for example, by 90 degrees, to the charged and set position of lancet 18c of FIGS. 15 and 16. The lancet cap 58 is keyed to the lancet body 52 so that when the user grasps and rotates the cap, the body rotates, too. Next, the user removes the lancet cap 58, for example, by manually pulling it off, so that the lancet tip 54 is now ready for use, as shown by lancet 18c of FIGS. 13 and 14.

The charging mechanism includes structures for retaining the lancet 18 in the charged and set position of lancet 18c of FIGS. 13 and 14. In a typical commercial embodiment, the charging mechanism includes two or another number of loading tabs 60 projecting from the lancet body 52 or elsewhere on the lancet 18. The loading tabs 60 slide along a linear or otherwise configured track 62 formed in or by the carrier 17 and/or the housing 12. When the lancet is pushed inward to the charged position of lancet 18a of FIGS. 15 and 16, the lancet body 52, and then the lancet cap 58, block the shutter 44 from closing. When the lancet is rotated to the charged and set position of lancet 18c of FIGS. 13 and 14, the loading tabs 60 extend into and are retained by loading recesses 64 formed in or by the carrier 17 and/or the housing 12. (The tracks 62 and recesses 64 can be seen in perspective in FIG. 11.) The lancet 18c of FIGS. 13 and 14 is now ready to be actuated by the actuator mechanism 14. It will be understood that alternatively the lancets 18 can be charged and set as part of the manufacturing process and shipped with all the lancets of the carousel 16 ready for use, or the user can charge and set the lancets before installing the carousel in the housing 12.

To actuate the charged lancet, the user operates the actuator mechanism 14 as described below, thereby rotating the lancet back to the position of lancet 18a of FIGS. 15 and 16 (except with the lancet cap already removed). The loading tabs 60 are thereby rotated back out of the loading recesses 64 so that the lancet is now free to move longitudinally with the loading tabs 60 sliding along the track 62. Then the charged operating spring 56 propels the lancet body 52 and tip 54 to the puncturing position of lancet 18b of FIGS. 13 and 14 to puncture the user's skin. The operating spring 56 then acts to pull the lancet back into the housing 12 to the shuttered position of lancet 18d of FIGS. 11 and 12.

In an alternative embodiment, the charging mechanisms include a ramped, curved, helical, and/or otherwise configured track assembly for rotating the lancet as it is pushed in to the charged position. In another alternative embodiment, the charging mechanisms include an automatic charging assembly for charging the next lancet automatically upon launching of the preceding lancet. In still another alternative embodiment, the charging mechanisms include another number and type of operating spring elements. In a further alternative embodiment, the charging mechanisms have the loading tabs extending from the carrier into a longitudinal track in the lancet body, and the lancet is rotated into a locked position with the tabs bearing on the front face of the lancet body. And in yet other alternative embodiments, the charging mechanisms include other structures for retaining the lancet in the charged position such as a latch, hook, catch, detent, and/or a spring-biased member, and/or the charged position and the charged and set position are the same, that is, the lancet is retained in place in the charged position.

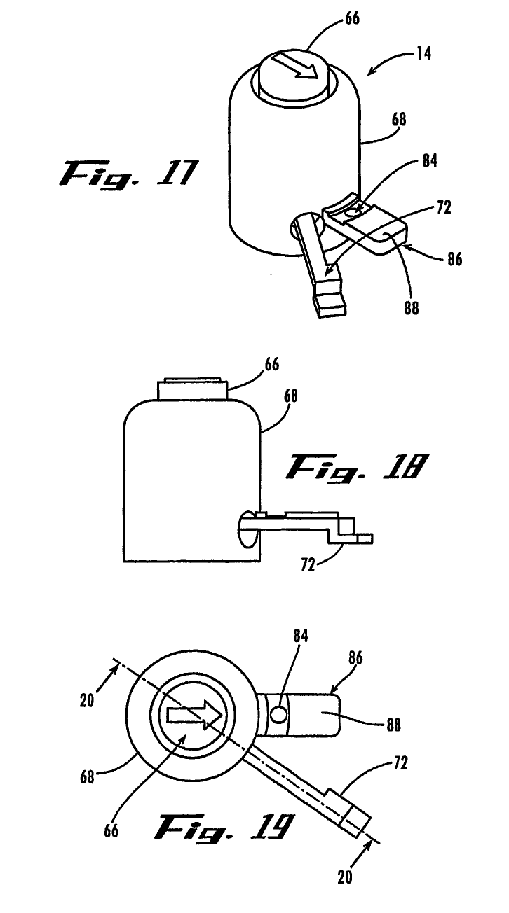

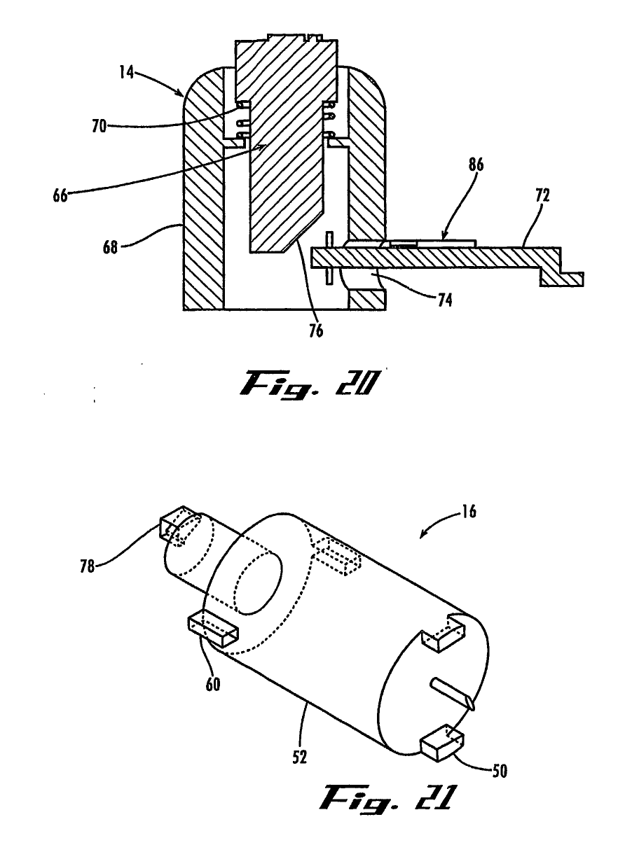

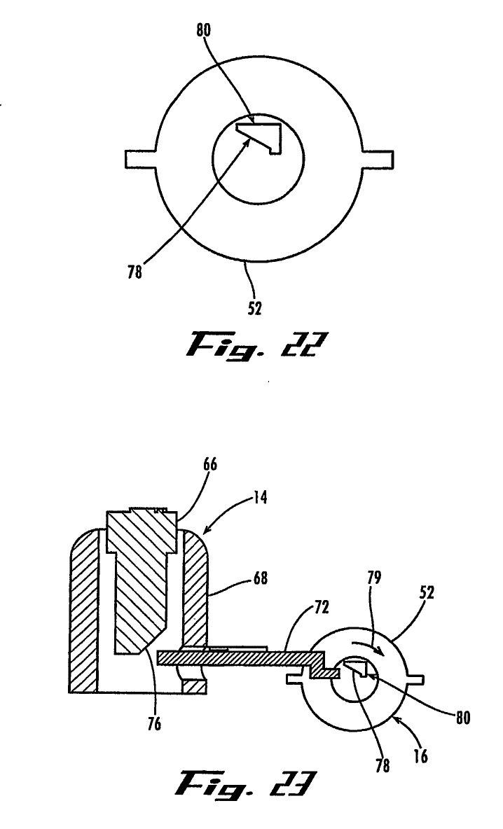

Referring now to FIGS. 17-23, there are illustrated details of the actuator mechanism 14 for launching the lancets 18. In a typical commercial embodiment, the actuator mechanism 14 includes an upright actuator button 66 that is received in an actuator housing 68 and is biased upward, for example by a spring element 70. In addition, the actuator mechanism 14 includes a lateral actuator arm 72 that extends through the actuator housing 68 and is biased inward, for example by a spring element 74. The actuator button 66 has an angled engagement surface 76 configured so that, when the user depresses the actuator button, the engagement surface 76 engages and moves the actuator arm 72 outwardly.

Each lancet body 52 has a cam surface 78 extending therefrom or formed therein that is engaged by the outwardly moving actuator arm 72. For example, the cam surface 78 may be formed on a wedge-shaped member 80 extending from the lancet body 52. Because of the shape of the cam surface 78, as the actuator arm 72 is further extended outwardly it rotates the lancet body 52 in the direction of arrow 79. As described above, when a charged lancet 18 is rotated, the loading tabs 60 are moved from engagement and retention by the loading recess 62, freeing the lancet to be launched by the charged operating spring 56. The actuator mechanism 14 is rotationally mounted to the central portion of the carrier 17 so that the user can rotate the actuator arm 72 into alignment with the peripherally arranged lancets 18 one at a time. Alternatively, the actuator mechanism may be rotationally mounted to the housing, instead of the carrier, so that it can be reused with multiple carousels.

In order to align the actuator mechanism 14 with a desired one of the lancets 18 for launching, one of several actuator alignment features may be provided. In a typical, commercial embodiment, the actuator alignment features include a detent mechanism for snap-locking the actuator arm 72 into operational alignment with the cam surface 78 of the lancet 18 to be actuated. For example, the alignment detent mechanism may include a series of bumps 82 on the housing 12 (see FIGS. 1 and 9) that can be received in a recess 84 on an alignment arm 86, or vice versa. In addition, the alignment arm 86 may have a flag portion 88 with an indicia such as a bright color marked thereon that is visible through an aligned window 90 in the housing 12 (see FIGS. 3 and 5). And a pointer such as the arrow shown on the actuator button 66 may also be provided.

In an alternative embodiment, the actuator arm includes the detent and flag, in other words, the features of the alignment arm are combined onto the actuator arm. In another alternative embodiment, the actuator alignment mechanism includes an automatic alignment mechanism for advancing and aligning the actuator mechanism with the next lancet automatically upon launching of the preceding lancet. And in yet another alternative embodiment, the actuator alignment mechanism includes another structure for securing the lancet in the proper alignment, such as a flexible or curved one-way catch and/or a spring-biased member.

To use the lancing device 10, the user inserts a fresh one of the carousels 16 into the housing 12, charges one of the lancets 18, actuates the charged lancet, and advances the actuator mechanism 14 to the next lancet. The charging, launching, and actuator advancing steps are repeated until all the lancets 18 of the carousel 16 have been used. Then the user removes and disposes of the spent carousel 16 and repeats the entire process.

The depicted exemplary embodiment is a lancing device comprising only lancing features. In alternate embodiments of the invention, the device includes sample collection features and/or sample analysis features. Accordingly, the invention includes an “all-in-one” device, comprising lancing features combined with both sample collection and analysis features.

In addition, the various features shown and described herein have all been presented in a single exemplary embodiment. It will be understood, however, that each of these features can be provided by themselves or in any combination in the lancing device described herein or in another multi-lancet or single-lancet lancing device.

While the invention has been described with reference to preferred and example embodiments, it will be understood by those skilled in the art that a variety of modifications, additions, and deletions are within the scope of the invention, as defined by the following claims.

What is claimed is:

1. A lancing device, comprising:
a housing;
at least one lancet having a tip that is movable between a puncturing position with the tip extending out of the housing and a retracted position with the lancet tip in the housing; and
at least one shutter mechanism including at least one shutter that is moveable between an open position not retaining the lancet from moving to the puncturing position and a closed position retaining the lancet in the retracted position.

2. The lancing device of claim 1, wherein the shutter mechanism further includes a shutter release member that holds the shutter in the open position.

3. The lancing device of claim 2, wherein the lancet is movable to a charged and set position from which it is actuated for launching to the puncturing position, and wherein the release member holds the shutter in the open position when the lancet is in the charged and set position.

4. The lancing device of claim 2, wherein the lancet further comprises a body from which the tip projects, and the shutter release member projects from the body of the lancet.

5. The lancing device of claim 4, wherein the shutter release member projects longitudinally from a front end of the body of the lancet.

6. The lancing device of claim 4, wherein the lancet body retains the shutter in the open position as the lancet moves from the charged position to the puncturing position.

7. The lancing device of claim 4, wherein the lancet further comprises a cap protecting the tip, and the lancet body and the lancet cap cooperatively retain the shutter in the open position as the lancet moves to the charged position.

8. The lancing device of claim 2, wherein the lancet is movable from a first position in which the shutter release member is in an engaged position retaining the shutter in the first position, to a second position in which the shutter release member is in a release position not retaining the shutter in the open position.

9. The lancing device of claim 8, wherein the lancet movement from the first position to the second position is rotational, and the shutter movement from the open position to the closed position is rotational.

10. The lancing device of claim 9, wherein axes of rotation of the lancet and the shutter are perpendicular.

11. The lancing device of claim 8, wherein the shutter is spring-biased to move from the open position to the closed position, and the shutter is free to move to the closed position when the lancet is in the retracted position.

12. The lancing device of claim 1, wherein the shutter moves from the open position to the closed position in response to the lancet being actuated to puncture the 'skin.

13. The lancing device of claim 12, wherein the shutter mechanism further includes a shutter release member movable to a release position not retaining the shutter in the open position, the lancet is actuated by rotation thereof for movement to the puncturing position, and the rotational actuation of the lancet rotates the shutter release member to the release position.

14. The lancing device of claim 1, further comprising a carousel having a carrier carrying a plurality of the lancets, wherein the carousel is removably received in the housing and the shutter is coupled to the carrier.

15. A lancing device, comprising:
a housing;
at least one lancet having a tip that is movable to a charged position with the lancet charged for launching, to a charged and set position with the lancet charged and retained in place, and to a puncturing position with the tip extending out of the housing; and
a charging mechanism including at least one loading recess and at least one loading tab that is rotated into and retained in the loading recess when the lancet is moved from the charged position to the charged and set position.

16. The lancing device of claim 15, wherein the loading tab is rotated out of the loading recess when the lancet is moved from the charged and set position to the charged position, to free the lancet for launching to the puncturing position.

17. The lancing device of claim 15, wherein the charging mechanism further includes at least one track that guides the loading tab when the when the lancet is moved to the charged position and from the charged position to the puncturing position.

18. The lancing device of claim 17, wherein the track is linear.

19. The lancing device of claim 15, further comprising a carousel including a carrier carrying a plurality of the lancets and a plurality of the charging mechanisms, the carousel removably received in the housing, the loading recess and the track defined by the carrier.

20. The lancing device of claim 15, wherein the lancet further comprises a body from which the tip projects, the loading tab projecting from the lancet body.

21. The lancing device of claim 15, wherein the charging mechanism further includes at least one operating spring that is charged when the lancet is moved to the charged position.

22. A lancing device, comprising:
a housing;
at least one lancet having a body and a tip extending from the body, wherein the lancet is rotatable from a charged and set position with the lancet charged and retained in place, to a charged position with the lancet charged and free to move to a puncturing position with the tip extending out of the housing; and
an actuator mechanism including at least one actuator arm that is movable into engagement with the lancet to rotate the lancet from the charged and set position to the charged position.

23. The lancing device of claim 23, wherein the actuator mechanism further includes a cam surface defined by the lancet or the actuator arm for converting linear movement of the actuator arm to the rotational movement of the lancet.

24. The lancing device of claim 23, wherein the cam surface is formed on a wedge-shaped member extending from the lancet body.

25. The lancing device of claim 22, wherein the actuator mechanism further includes an actuator button that is biased away from the actuator arm and is moveable into engagement with the actuator arm to move the actuator arm linearly into engagement with the lancet.

26. The lancing device of claim 25, wherein the actuator mechanism further includes an angled engagement surface defined by the actuator button or the actuator arm for engaging to move the actuator arm.

27. The lancing device of claim 22, further comprising a carousel having a carrier carrying a plurality of the lancets, the carousel removably received in the housing, and the actuator mechanism rotationally coupled to a central portion of the carrier for selectively operably aligning the actuator arm with the lancets.

28. The lancing device of claim 27, wherein the actuator mechanism further includes an alignment arm with a mark thereon, the housing defines a plurality of windows corresponding to the lancets, and the alignment arm mark is visible through the window corresponding to the lancet that the actuator arm is aligned with.

29. A lancing device, comprising:
a housing;
a plurality of lancets; and
a carousel that is removably received in the housing and includes a carrier that is adapted for carrying the lancets in a non-radial configuration.

30. The lancing device of claim 29, wherein the carrier is adapted for carrying the lancets in a tangential configuration.

31. The lancing device of claim 29, wherein the housing has a plurality of protrusions that correspond to the lancets, with each of the protrusions having a surface generally perpendicular to a puncturing stroke direction of a corresponding one of the lancets.

32. The lancing device of claim 31, wherein the protrusions are generally wedge-shaped.

33. The lancing device of claim 31, wherein the lancets each have a puncturing tip, the housing defines a plurality of openings through which corresponding ones of the lancet tips are extendable, and the lancet openings are formed in the perpendicular surfaces.

34. The lancing device of claim 29, wherein the housing includes recesses for receiving and guiding the lancets.

35. The lancing device of claim 29, further comprising a lock mechanism including at least one key-tabbed member and at least one keyed opening that matingly receives the key-tabbed member, wherein the key-tabbed member and the key-opening are configured so that the carousel can be rotated into a locked position.

36. The lancing device of claim 35, wherein the keyed opening comprises a keyhole or a curved key-slot.

37. The lancing device of claim 35, wherein the key-tabbed member extends from the housing and the keyhole or the keyed opening is defined in the carrier.

38. The lancing device of claim 35, wherein the lock mechanism further includes at least one detent bump that is alignable with the keyed opening.

39. The lancing device of claim 29, further comprising a lock mechanism including at least one threaded member and at least one matingly threaded hole that receives the threaded member to secure the carousel in the housing.

40. A lancet carousel for a lancing device including a housing, the carousel comprising:
a plurality of lancets; and
a carrier that is adapted for carrying the lancets in a non-radial configuration, wherein the carousel is removably receivable in the housing.

41. The lancet carousel of claim 40, wherein the carrier is adapted for carrying the lancets in a tangential configuration.

42. The lancet carousel of claim 40, wherein the lancing device includes at least one key-tabbed member, and wherein the carrier includes at least one keyed opening that matingly receives the key-tabbed member and is configured so that the carousel can be rotated into a locked position secured to the housing.

43. The lancet carousel of claim 40, wherein the lancing device includes at least one threaded member, and wherein the carrier includes at least one threaded hole that matingly receives the threaded member to secure the carousel in a locked position in the housing.

44. A method of lancing skin, comprising:
a) providing a lancing device having a housing and a lancet carousel, the lancet carousel including a plurality of lancets carried by a carrier in a non-radial configuration;
b) inserting the carousel in the housing;
c) charging one of the lancets;
d) launching the charged lancet;
e) advancing the actuator mechanism from one of the lancets to a next one of the lancets;
f) repeating steps (c) through (e) until all the lancets are actuated; and
g) removing the carousel.

45. The method of claim 44, wherein the step of charging one of the lancets includes grasping a sterility cap of one of the lancets, pushing the lancet inward so that at least one loading tab is guided by a track to guide the lancet to a charged position, and rotating the lancet so that the loading tab is received in at least one loading recess to retain the lancet in a charged and set position.

46. The method of claim 45, further comprising the step of removing the sterility cap after the charging step and before the launching step.

47. The method of claim 44, wherein the step of launching the charged lancet includes actuating an actuator mechanism to engage and rotate the lancet back to the charged position so that the loading tab is removed from the loading recess and the charged lancet is free to be launched.

48. The method of claim 44, wherein the step of advancing the actuator mechanism includes rotating an actuator arm of the actuator mechanism from operable alignment with the one lancet to operable alignment with the next lancet.
